(19) United States
(12) Patent Application Publication
(10) Pub. No.: US 2013/0274840 A1
(43) Pub. Date: Oct. 17, 2013

(54) DEVICE FOR PERSONAL HEATING USING A DIRECTED ENERGY BEAM

(71) Applicant: The Research Foundation of State University of New York, Binghamton, NY (US)

(72) Inventor: Kenneth James McLeod, Vestal, NY (US)

(21) Appl. No.: 13/864,792

(22) Filed: Apr. 17, 2013

Related U.S. Application Data
(60) Provisional application No. 61/625,360, filed on Apr. 17, 2012.

(51) Int. Cl. A61F 7/00

(52) U.S. Cl. CPC A61F 7/00
USPC 607/100

(57) ABSTRACT

The present disclosure describes improvements to personal heating that can raise the temperature of a target (e.g., a human) to maintain comfort, but at much less operating costs of conventional heating devices, e.g., space heaters. The embodiments describe devices (and system and methods) that utilize an energy beam (e.g., having a wavelength in the infrared spectrum) that changes the temperature of the outer layers of skin on a human. These embodiments offer an individualize solution to personal heating at relatively low energy consumption.

### CROSS-REFERENCE TO RELATED APPLICATIONS

This application claims priority to U.S. Provisional Patent Ser. No. 61/625,360, filed on Apr. 17, 2012 and entitled “Heating Device, System, and Method.” The content of this provisional application is incorporated by reference herein in its entirety.

### BACKGROUND

The present disclosure describes subject matter that relates to heating and cooling, and in several embodiments, to personal heating technology that employs electromagnetic energy to change the temperature of a target (e.g., a human) to maintain heat balance and/or to promote weight loss.

Heating and cooling of office space and homes often requires large industrial systems (e.g., heating, ventilation, and air conditioning (HVAC) systems). The design of these systems offers economies of scale to regulate temperature in many different areas from a single (and/or multiple) source. However, although temperature regulating systems are meant to maintain conditions at certain comfortable levels, it is rare for any system to operate in a manner that results in environments that match certain optimal conditions that are most comfortable for the individuals residing and/or working therein. Thus, to achieve optimal and/or individualized conditions, many individuals must deploy individual heating units and, more likely, space heaters to stay warm and comfortable. These units deliver warm air directly onto the end user. But operation of these space heaters can increase electricity costs. For example, a typical 1,200 W-1,500 W space heater can cost upwards of $2 per workday per employee, which can increase energy bills on the order of $600 per employee during the work year.

### BRIEF DESCRIPTION OF THE INVENTION

The present disclosure describes improvements to personal heating that can raise the temperature of a target (e.g., a human) to maintain comfort, but at much less operating costs of conventional heating devices, e.g., space heaters. The embodiments below describe devices (and system and methods) that utilize an energy beam (e.g., having a wavelength in the infrared spectrum) that changes the temperature of the outer layers of skin on a human. These embodiments offer an individualize solution to personal heating at relatively low energy consumption.

These improvement address, inter alia, weight-related issues that precipitate from heat imbalance that result from residing, working, and/or operating in environments that are at less-than-optimal conditions. Approximately two-thirds of the workforce in the United States has been estimated to be overweight or obese, and the direct and indirect costs associated with an overweight workforce have been established at over $150B USD per year, or approximately $1,000 USD per employee per year. These costs include increases in worker sick days and treatment for disease, e.g., diabetes and heart disease. As set forth more below, the embodiments below can help employees to effortlessly lose unwanted weight and, thus, improve the emotional and physical health of employees. These benefits coincide with improvements in worker productivity and reductions in healthcare costs.

The embodiments below find use in numerous other applications beyond the workplace. The improvements in personal heating and thermal/temperature control technology may significantly improve the health and ability to recover from illness or injury for elderly and infirm individuals who often have difficulty staying warm, even in environments where young, healthy individuals are quite comfortable. These embodiments can also install into homes of individuals to obtain the same results as apply to the workplace, e.g., to reduce home heating costs, enhance personal comfort, and the like. Personal heating and thermal/temperature control that these embodiments offer may further help to maintain body temperature in individuals that operate in environments where conventional heating technologies are impractical. The environments include, for example, very cold environments (e.g., loading docks, refrigeration units, etc.) and outdoor occupations (e.g., outdoor repairmen, linemen, construction, etc.).

### BRIEF DESCRIPTION OF THE DRAWINGS

Reference is now made briefly to the accompanying drawings in which:

FIG. 1 depicts a schematic diagram of an exemplary embodiment of a heating device that irradiates a target with an energy beam;

FIG. 2 depicts a schematic diagram of a side view of an exemplary embodiment of a heating device;

FIG. 3 depicts a front view of the heating device of FIG. 2;

FIG. 4 depicts a schematic diagram of an exemplary embodiment of a heating device as part of a system.

FIG. 5 depicts a side view of an exemplary embodiment of a heating device to illustrate one exemplary form factor;

FIG. 6 depicts a flow diagram of a method for changing temperature of an individual;

FIG. 7 depicts a flow diagram of a method for inducing weight loss in an individual;

FIG. 8 depicts a plot to illustrate heat imbalance among adult women working in an office environment;

FIG. 9 depicts a plot to illustrate the dependence of growth hormone (GH) levels in the blood (in young adults) as a function of core body temperature;

FIG. 10 depicts a plot to illustrate the response of core body temperature to an environmental perturbation, e.g., exposure to an energy beam; and

FIG. 11 depicts a plot to illustrate the change in body mass over time for adult women under one embodiment of a method for inducing weight loss.

Where applicable like reference characters designate identical or corresponding components and units throughout the several views, which are not to scale unless otherwise indicated.

### DETAILED DESCRIPTION

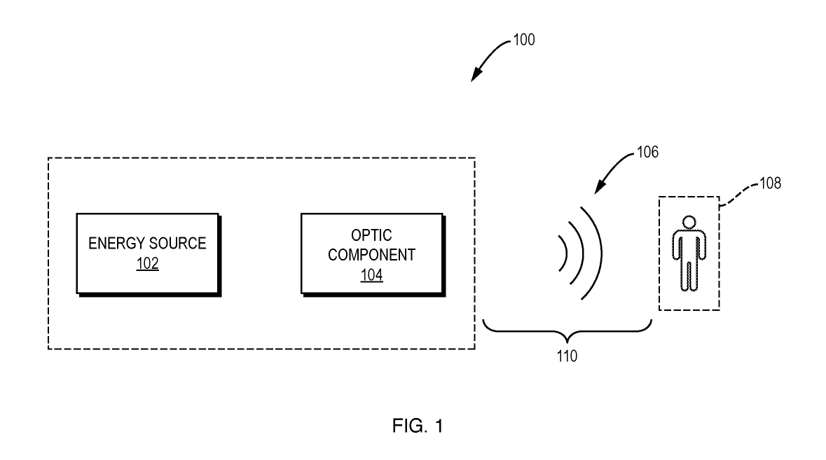

FIG. 1 depicts a schematic diagram of a heating device 100 that can raise the temperature of an individual over a distance. The heating device 100 includes an energy source component 102 and an optics component 104. This combination of components generates an energy beam 106 that can irradiate a target 108 (e.g., a human) spaced apart a distance 110 from the heating device 100.

Broadly, embodiments of the heating device 100 generate the energy beam 106 with beam parameters (e.g., wavelength) that can change the temperature of the target 108. For use with humans, the heating device 100 can help regulate heat transfer that can alter the core temperature of the body. In one embodiment, the energy beam 106 has beam parameters that raise the temperature of the outer layers of skin tissue (e.g., the epidermis) to generate heat energy that dissipates throughout the body. This heat energy remediates improper, or negative, heat imbalances that can result when the body loses heat (e.g., via dissipation through the skin) at a rate that exceeds heat energy the body generates by metabolic activity. In one example, use of the heating device 100 can remediate negative heat imbalances in a range from about 5 W to about 30 W.

The heating device 100 can also stimulate certain physiological responses that facilitate weight loss in humans. Exposure to the energy beam 106, for example, can raise the core temperature of the body to levels that promote production of growth hormone. This feature can help regulate body mass and, in one implementation, reduce weight. In one example, use of the heating device 100 can integrate into a treatment method (also, protocol) that exposes the human to the energy beam 106 for a set time period (e.g., 30 minutes) and/or at a pre-determined periodic treatment interval (e.g., 3 days/week). This treatment method can implement the heating device 100 passively, e.g., by operating the heating device 100 to maintain heat balance in work and/or home setting, and/or actively, e.g., as part of a fitness/weight loss regime.

Examples of the energy source 102 include lasers and related light amplification devices. These devices can have various constructions (e.g., gas, chemical, infrared (IR) laser diode, etc.) to generate the energy beam 106. For example, the devices can have a power rating in a range from about 10 W to about 50 W, although this disclosure contemplates implementations in which the power rating is about 50 W or greater. In one embodiment, the energy source 102 comprises a carbon-dioxide (CO₂) laser. Construction of the energy source 102 can generate one or more energy beams (e.g., energy beam 106) with beam parameters that can heat the body without adverse affects (e.g., burns, etc.). The beam parameters may define a wavelength, which may identify the position and/or location of the energy of the energy beam, e.g., on the electromagnetic energy spectrum. Examples of the energy beam 106 may have a wavelength found in a range of about 3 micrometers or greater and/or as infrared and/or far infrared relative to the electromagnetic energy spectrum. In one example, the wavelength defines infrared-C (IR-C) energy.

The optics component 104 can diffuse and direct the energy beam 106 from the energy source 102. Examples of the optics component 104 can include one or more lenses and/or lens elements that can transmit the energy beam 106. These elements may exhibit diffusive, transmissive, refractive, and/or reflective properties (e.g., speckled surfaces and similar diffusive surfaces). Moreover, these elements may have physical characteristics (e.g., shapes, contours, form factors, and like) that can manipulate the energy beam 106 as desired. For example, construction of the optics component 104 may adjust the size and/or shape of the energy beam to cover a certain region and/or area on the target 108.

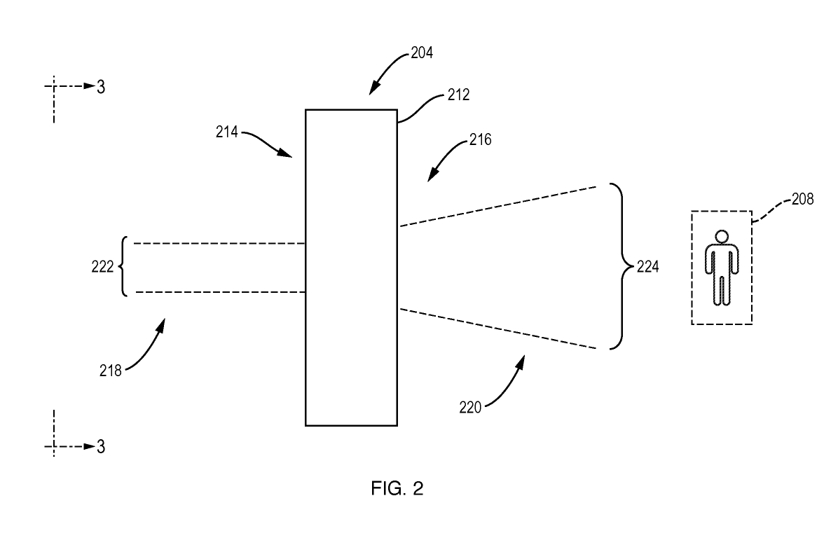

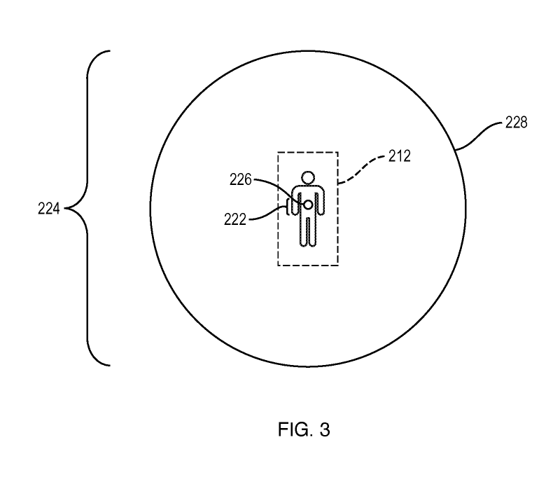

FIGS. 2 and 3 depict schematic views of another exemplary embodiment of a heating device 200 to illustrate operation of the optics component 204 to modify the energy beam 206. As shown in FIG. 2, the optics component 204 includes one or more lens elements (e.g., a first lens element 212) that has a first side 214 and a second side 216 proximate the target 208. The energy beam (e.g., energy beam 106 of FIG. 1) includes a first beam 218 and a second beam 220 that exhibit, respectively, one or more beam configurations (e.g., a first beam configuration 222 and a second beam configuration 224). As shown in FIG. 3, the first beam configuration 222 and the second beam configuration 224 can define, respectively, a first coverage region 226 and a second coverage region 228 that is different from the first coverage region 226. The beam configurations 222, 224 define features of the energy beams 218, 220. These features include, for example, shapes and dimensions (e.g., length, width, radius, diameter, etc.). Exemplary shapes include annular, circular, and elliptical shapes, although this disclosure contemplates configurations of the first lens element 212 that can modify the energy beam 206 to accommodate any variety of shapes and sizes as desired. Moreover, although the shapes and sizes can vary, in one embodiment, the first lens element 212 is configured to generate the shape of the second coverage region 228 with an area of about 500 cm² or greater at the target, and in one implementation, the area is about 2000 cm² or greater. In one example, the area is about 2700 cm². Selection of the area may also correspond to the size of a portion of a human that is irradiated by the energy beam. For example, the area may be sized and configured to cover the head, torso, and/or other parts of the human body, as well as combinations thereof.

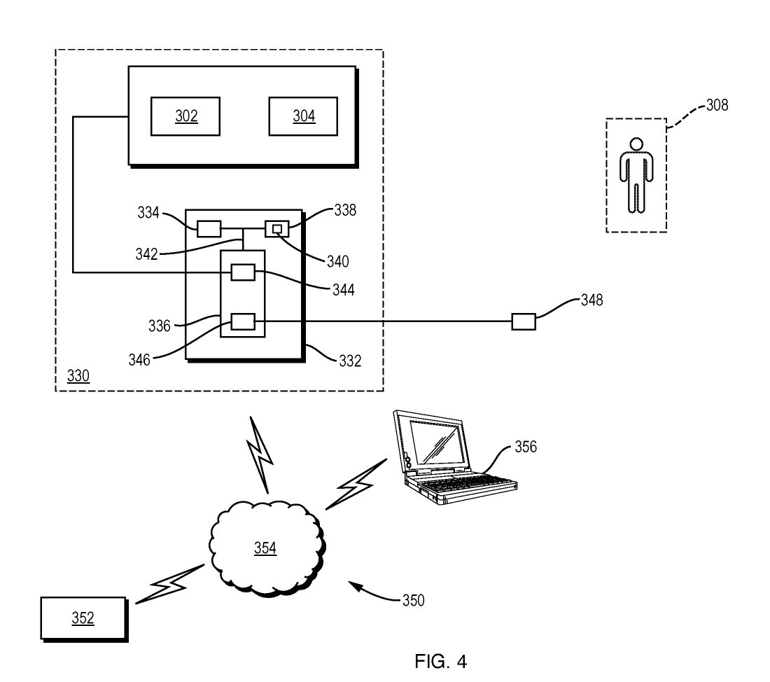

FIG. 4 illustrates a schematic diagram of an exemplary heating device 300 as part of a system 330 (also, “control system 330”). Examples of the system 330 may be incorporated into homes, offices, and like buildings and structure. Although not shown, the system 330 may integrate with existing heating and cooling systems, e.g., heating, air conditioning, and ventilation (HVAC) systems to regulate temperature of individuals that are operating in the spaces of the building.

As shown in FIG. 4, the system 330 includes a control device 332 that can generate signals to instruct operation of the heating device 300. Examples of control device 332 can include a remote control that communicates with the heating device 300, e.g., by way of wireless signals, protocols, and the like. In one embodiment, the control device 332 has a processor 334, control circuitry 336, and memory 338, which can store one or more executable instructions 340, e.g., in the form of software and firmware that are configured to be executed by a processor (e.g., the processor 334). The control device 332 can also includes busses 342 to couple components (e.g., processor 334, control circuitry 336, and memory 340) of the control device 332 together. The busses 342 permit the exchange of signals, data, and information from one component of the control device 332 to another. In one example, control circuitry 336 includes a device driver circuit 344 and a sensor driver circuit 346. The device driver circuit 344 couple with the heating device 300 to convey signals that instruct operation, e.g., of the energy source 302. The sensor driver circuit 346 can couple with one or more sensor elements (e.g., a first sensor element 348) that can provide signals to the control device 332. These signals may define a value for a target response parameter, which may help to instruct operation of the heating device.

Examples of the target response parameter can include conditions of the target (also “target conditions”) as well as conditions of the environment surrounding the target (also “environmental conditions”). The sensor element may also be responsive to the ambient environment surrounding the target 308. Examples of the sensor element 348 may sense and/or measure temperature, relative humidity, and other factors that can affect the temperature, e.g., of a human.

The target conditions may include, for example, temperature of the target (e.g., core temperature, temperature of an outer layer of skin, etc.). In one example, the sensor element is a temperature sensor that is disposed on the target to monitor temperature of the skin. The target conditions can also include physiological responses of the target, e.g., levels of human growth hormone. These physiological responses may identify one or more biochemical response of the target at specific temperature (e.g., a second temperature that is higher than a first temperature of the target). In one implementation, the physiological response relates to weight loss and/or weight gain in a human that is subject to irradiation by the energy beam. For example, the physiological response can identify a change in weight of the human from a first weight to a second weight that is different from the first weight.

The target conditions may also include a clinical response of a human. Examples of the clinical response may measure certain parameters of the human in a surgical setting and/or other clinical setting when temperature of the human is modified by irradiation by the energy beam. The clinical response may, for example, measure neurological activity of the human, and the like. In another example, the sensor element comprises a device that couples with the human to record electrical acidity of the human that indicates the neurological activity, e.g., for performing and/or recording data in connection with electroencephalography (EEG). This disclosure contemplates any number of devices for use as the sensor element that can provide data for purposes of regulating temperature of the target via the heating device 300. This data may, for example, identify a comfort level and/or provide other indicators of the comfort of an individual, which can prompt the individual to modulate the energy beam, e.g., by turning the heating device on and/or off. This operation can be done via the remote control. Other forms of modulation can adjust parameters of the energy beam and/or operation of the heating device, e.g., to gradually reduce power input to change the temperature of the skin of a human.

The control device 332 can communicate with a network system 350 with one or more external servers (e.g., external server 352) and a network 354 that connects the control device 332 to the external server 352. This disclosure also contemplates configurations in which one or more programs and/or executable instructions are found on the external server 352. The control device 332 can access these remotely stored items to perform one or more functions disclosed herein. In one embodiment, a computing device 356 may communicate with one or more of the control device 332 and the network 354, e.g., to interface and/or interact with the heating device 300 and/or system 330, as desired.

At the system level, the control device 332 can instruct operation of the heating device 300 to regulate operation of the energy source 302 and/or the optics component 304. Use of the control device 332 and sensor element 348, for example, can create a feedback loop that monitors conditions proximate the target 308 to select appropriate parameters for the energy beam, to turn the beam on/off, as well as other operations that will help modulate exposure of the target 308 to the energy beam. Many of these features may be automated and/or otherwise have configurations that can tailor and/or modify the coverage in response to inputs. Exemplary inputs can arise from the sensor element 348, as discussed, and/or from an end user (e.g., target 308), and/or via a remote system that integrates with the building and/or dwelling that deploys the heating device 300 and/or the system 330. As set forth above, the inputs can also arise from various types of sensor elements and devices that monitor target conditions and/or environmental conditions.

The control device 332 can help to facilitate these types of controls. The control devices 332 can comprise various types of discrete electrical devices that include processors and memory. The control device 332 can also comprise various control circuitry to drive, operate, and manage the overall function of the heating device 300 and/or system that incorporates the heating device 300. Examples of the control device 332 can also include various types and compilations of executable instruction (e.g., software and or firmware instructions and programs), which can be stored on memory and are configured to be executed by the processor. In some examples, the control device 332 can interface with one or more peripheral devices including sensors, e.g., temperature sensors that couple with the target 308 to provide an input that relates to the temperature of the target 308. Other peripheral devices can include computing devices (e.g., laptops and desktop computers), databases, hand held computing devices (e.g., smartphones, tablet computers, etc.). In still other examples, the control device 332 can couple with various types of temperature control systems (e.g., HVAC systems) that may include thermostats and like devices that facilitate thermal control of the environment in large spaces (e.g., offices, office buildings, homes, rooms, etc.).

In other implementations, the system 330 can operate to coordinate operation of the heating device 300 with the movement of the target 308, e.g., to maintain irradiation of a human moving about a room. This feature may utilize tracking systems and/or sensors that can generate signals with data to identify the position of the target 308 relative to the heating device 300 and/or locations in the room. In other implementations, the system 300 is configured to irradiate multiple targets 308. This feature can be accomplished using a plurality of heating devices 300 and/or certain configurations of optics components and/or combinations thereof to generate energy beams to irradiate one or more people.

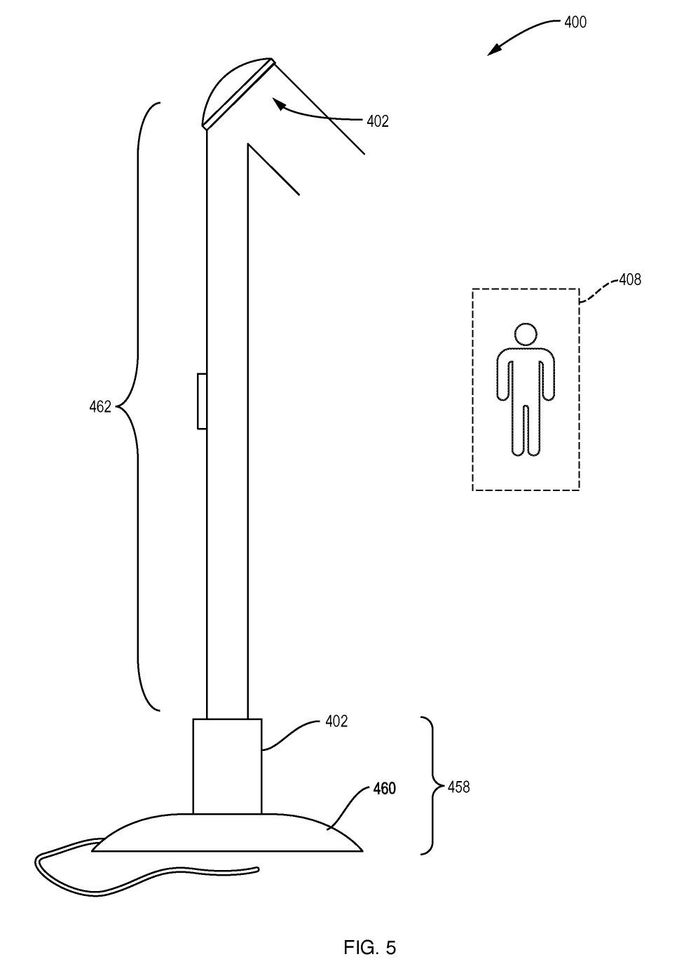

FIG. 5 depicts an exemplary form factor for an exemplary embodiment of a heating device 400. This form factor embodies a stand-alone unit, similar in one or more aspects to a floor or desk lamp. In other examples, the general structure of the heating device can take a form factor conducive with surgical and/or operating rooms, wherein the heating device 400 can irradiate patient on an operating table.

As shown in the example of FIG. 5, the heating device 400 can comprise a base component 458 with a support 460 and, in one example, the energy source 402, e.g., a CO₂ laser. The heating device 400 can also comprise an elongated structure 462 with a head component 464 that houses the optics component 404. Examples of the elongated structure 462 can direct energy from the energy source toward the diffuser, which can expand and redirect the energy beam towards the target, e.g., an individual that requires supplemental heating.

Due to the compact nature and high power output of exemplary CO₂ lasers, examples of the heating device 400 can operate as a personal radiant heat system with output to the target 408, e.g., in far-infrared range. These types of lasers are easy to operate in pulsed mode, allowing for radiant energy levels to be controlled by the user.

Humans often prefer irradiation from a non-symmetric radiant heat source to occur from their front or back, rather than from the floor or ceiling. To this end, embodiments of the heating device 400 (and heating devices 100, 200, 300 of FIGS. 1, 2, 3, and 4) may embody form factors that devise a “floor lamp” design (as shown in FIG. 5) wherein the source is about 2 m above the floor permitting it to be aimed downward toward the head and chest of the user; a desk lamp type device which could radiate either downward toward the user, or upward toward the user, if, for example it were placed below a computer monitor; and/or a ceiling mounted device that could readily incorporate tracking technology so that as a person moved around a room, the radiant power output could be continuously adjusted to maintain the desired comfort level.

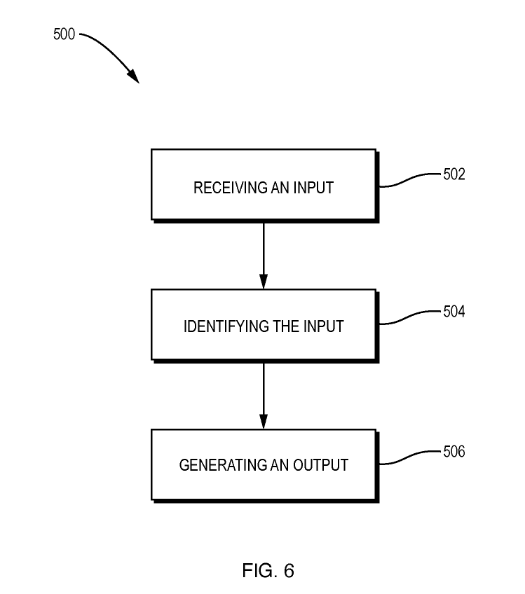

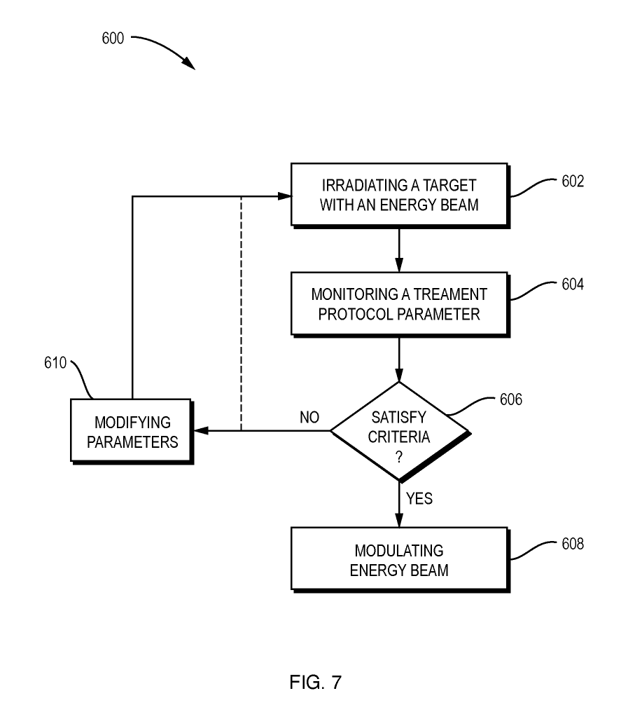

FIGS. 6 and 7 depict flow diagrams of a method 500 (FIG. 6) and a method 600 (FIG. 7) that can regulate temperature of a target (e.g., a human) to remedy heat imbalance and/or to promote weight loss. Broadly, the steps of the method 500 and the method 600 may embody one or more executable instructions, which can be coded, e.g., part of hardware, firmware, software, software programs, etc.) that, when executed, can cause the heating device and/or related system to generate energy beams with various properties and configurations. These executable instructions can be part of a computer-implemented method and/or program, which can be which can be stored on memory (e.g., memory 338 of FIG. 4) and executed by a processor (e.g., processor 334 of FIG. 4) and/or processing device.

As shown in FIG. 6, the method 500 includes, at step 502, receiving an input, at step 504, identifying the input and, at step 506, generating an output in response to the input. In one embodiment of the method 500, the input can comprise one or more electrical signals from a sensor (e.g., sensor element 348 of FIG. 4) and/or from an accompanying system or peripheral device. These inputs can instruct operation of the heating device, e.g., to change one or more properties of the energy beam, to change the beam configuration (including the shape, size, area of coverage, etc.). When the input comprises temperature, for example, the method 500 may include one or more steps for identify the input as temperature and for comparing the value of the temperature to a threshold value, e.g., that defines the desired temperature for the target. Likewise, the input may on the other hand comprise electrical signals that are instructive as to temperature and other functions, e.g., signals from a remote control that turn the heating device on/off and/or that changes other operating parameters of the heating device. Any one of these inputs may result in an output (e.g., at step 206). Exemplary outputs may change the operation of the energy source, as well as activate features of the optics component to change, modify, or alter the parameter of operation of the heating device.

In FIG. 7, the method 600 includes, at step 602, irradiating a target (e.g., a human) with an energy beam. As noted herein, exposure to the energy beam can change the temperature of the target, e.g., from a first temperature to a second temperature that is higher than the first temperature. The method 600 also includes, at step 604, monitoring a value for a target response parameter. The method 600 also includes, at step 606, comparing the value to a threshold criteria for the target response parameter. If the treatment protocol parameter satisfies the treatment value, the method 600 can continue, at step 608, modulating the energy beam. This step may, in one example, cease exposure of the target to the energy beam, e.g., where power of the energy beam is modulated to at and/or near zero. On the other hand, if the treatment protocol parameter does not satisfy the threshold criteria, the method 600 can return to step 602 to maintain irradiation of the target and/or, in one embodiment, the method 600 includes, at step 610, modifying parameters (e.g., power, wavelength, etc.) of the energy beam.

In one embodiment, the method 600 may further include one or more steps for collecting a first body indicator of the target and comparing the first body indicator to a baseline value. Examples of the first body indicator and the baseline value may define a weight for a human, a percent body fat for the human, as well as other parameters that can gauge weight loss and/or weight gain for the human. In one example, the target parameter and the threshold criteria can define an exposure parameter for irradiation of the human. This exposure parameter can define a period of time that the human is subject to irradiation. The period of time can measure seconds, minutes, hours, days, weeks, and the like. In one example, the relative position of the first body indicator relative to the baseline value may define the value for the exposure parameter, e.g., by defining the length of time that the human undergoes to achieve a certain, desirable weight.

The steps of the method may be applied to a weight loss protocol and/or treatment to expose and individual to the irradiation by the energy beam to achieve a certain, desired weight. This exposure can occur for a defined period of time and/or for a set number of exposures over a day, week, month, year, etc. The target response parameter may identify one or more responses of the human at a temperature (e.g., the second temperature). As set forth herein, these responses may include physiological response and clinical responses, e.g., that can indicate a need to change the temperature of a patient during surgery. Use of the devices, systems, and method described herein can allow personal heating of the patient independent of the temperature of the operating room.

### Experimental Examples

In light of the foregoing discussion, implementation of the embodiments contemplated herein may find particular use with heating and temperature regulation of individuals. Humans are homeotherms with physiologic systems that help to maintain core body temperature at about 30° C. or greater, and in one example, in a range from about 32° C. to about 44° C. despite wide variations in physical activity levels and environmental conditions (e.g., temperatures and humidity levels). Below temperatures of about 35° C., many systems in the body function poorly. On the other hand, temperatures above about 40° C. increase risks of neuronal damage, particularly in the brain. For individuals who are not doing manual labor (e.g. the typical office employee) the most comfortable indoor environment is in a range of about 25° C. to about 27° C. at a relative humidity of 30%-40%. These conditions serve to keep the skin temperature in a range of about 32° C. to about 34° C. with minimal sweat accumulation on the skin (wet skin is strongly correlated to environmental discomfort).

Unfortunately, buildings rarely achieve, or maintain, conditions within this optimal range of temperatures and/or relative humidity. Across the United States, buildings in cool climates must warm entry air; and in southern climates, wherein many states have an average relative humidity over 40%, buildings must chill entry air below the dew point to remove moisture and then reheat the air to appropriate temperatures. Heating and reheating are expensive and, thus, building temperatures often maintain conditions at a minimally acceptable levels, typically about 21° C. and 40%-50% relative humidity. However, individuals (e.g., employees) that spend time under these conditions can become physically uncomfortable. These individuals may complain of cold hands and/or cold feet. For businesses, there are even more direct and indirect costs that these less-than-optimal conditions can cause, including:

Reduced Productivity:

Employees who are physically uncomfortable tend to be far less productive. For example, a study from Cornell University found that workers in a room at a temperature of about 20° C.—a temperature recommended by the federal government to conserve energy—perform monotonous keyboarding tasks 54% of the time with a 25% error rate. Raising the temperature to about 25° C., workers worked 100% of the time with a 10% error rate, more than doubling productivity.

Increased Costs:

Employees may employ conventional heating devices (e.g., electric space heaters) to stay warm and comfortable. A typical space heater is rated 1200 W-1500 W devices (power being consumed). Thus, at a delivered electricity cost of $0.15 per KW-Hr, operation of these conventional heating devices can cost over $2.00 USD per workday per employee. During a typical work year, these additional charges can amount to $600.00 USD per employee depending on the needs of the employee and other variables, e.g., the geographic region, climate variables, electricity costs, and the like. In a region with high electricity costs, e.g., Long Island, N.Y., a small office with 30 employees can incur annual costs to supplement heating in excess of $30,000 USD per year.

Reduced Efficiency:

Building often utilize systems that heat all spaces to a common temperature, whether the spaces are occupied or not. This type of construction is not a “green” building. As heating and cooling accounts for close to 45% of all energy use in the U.S., inefficient use of energy is not sustainable, and will not be acceptable in the future. Building managers should consider the sustainability of their operations, i.e., to keep people warm and comfortable, while the structure and equipment are kept cool.

Reduced Health:

Humans adapt to stressful environments, thus chronic exposure to less-than-optimal conditions, e.g., chilled environments, may result in adaptation over time to ensure the body can maintain core temperature. An example of one adaptation is weight gain, which is a natural response of the body to both insulate and to increase internal heat production. Research has shown that all of the weight gain observed in adult men over the past 40 years in the United States, and 80% of the weight gain observed in adult women over the same time period, can be attributed to the fact that many Americans spend most of living and working days in chilled environments (e.g., air conditioned). The increase in body weight, which can progress to obesity, may result in significant increases in healthcare expenses.

Personal heating and related thermal/temperature control technology (e.g., use and implementation of heating devices 100, 200 of FIGS. 1, 2, 3, 4, and 5) can address many of the challenges above. Moreover, while immediate applications of this technology can enhance work spaces, longer term, and much larger, applications abound in new construction, where implementation of technology can reduce building heating and air conditioning costs. In addition, there is the potential to make this technology “smart”—that is, for example, by converging RFID or other information transfer processes to allow an individual (e.g., an employee) to “signal” their comfort level to a supplemental radiant heating system that deploys heating devices of the present disclosure throughout the building. This feature can accommodate variations in environment from individual-to-individual to remedy heat imbalance. For example, the system could allow individuals in the same room to adjust exposure to radiant heating (e.g., energy beam 106, 206) to maintain the individual at a preferred body temperature, independent of the temperature of the room.

Metabolic activity in humans, under typical working conditions (i.e. not manual labor), result in heat generation per kilogram of lean body mass in a range of about 1 W to about 1.5 W. Under these conditions, a typical individual generates about 50 to about 200 Watts (Joules/s) of heat even when working in an office. The body must lose this heat in order to maintain core temperatures at relatively constant values.

The body utilizes a variety of heat transfer mechanisms to lose this heat. Notably, these mechanism are all surface dependent processes. There are four principle heat loss mechanisms; conduction, convection, evaporation/condensation, and radiation. Conductive heat transfer is rather low, with exceptions for workers that labor outdoors in a wet environment and, therefore, experience significant heat transfer by conduction. Similarly, in the absence of high air velocities, convective heat transfer contributes very little to heat gain or loss. Therefore, in indoor environments, the dominant modes of heat transfer to and from the body are evaporative heat loss and radiation (although, condensation heat gain also plays a relatively insignificant role). Evaporative heat losses occur during breathing, as saturated air in the lungs is exhaled. Sweating also permits remarkably high heat transfer rates. However, in an office environment, sweating generally amounts to perhaps 10% of total heat loss. Accordingly, approximately three-quarters of all heat loss and/or environment heat gain from the body arises through radiation. For example, to remove the metabolic heat, the body radiates heat from the skin surface to our surroundings, and the rate at which we radiate heat is a function of the temperature of the surroundings, the temperature of the skin, and the presence of clothing and/or materials about the skin.

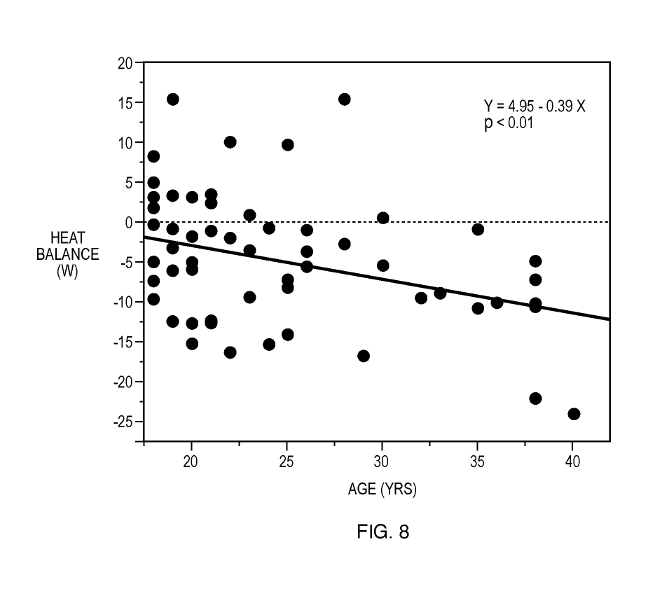

With reference to FIG. 8, humans are often most comfortable when the skin is at a temperature in a range of about 32° C. to about 34° C. If the environment surrounding the body is at a temperature of about 25° C. to about 27° C., the body can effectively radiate about 50 to about 200 Watts, which corresponds to the energy metabolic activity generates to keep the body in heat balance. On the other hand, if the environment surrounding the body is cooler, for example, at a temperature of about 21° C., heat loss may exceed metabolic heat generation and an individual will be in negative heat balance. This condition can cause an individual to feel physically uncomfortable (e.g., cold hands, cold feet, etc.) and, moreover, may require supplemental heating. In an office work environment at about 21° C., many individuals are in a net negative heat balance and, for example, exhibit a heat imbalance in a range up to about −25 W, with imbalance increasing with the individual's age.

As set forth above, radiant energy can warm the body to remediate these imbalances. The skin can absorb radiant energy over a wide range of wavelengths and, notably, absorb is close to 100% for radiant energy at wavelengths that are about 3 micrometers (i.e. there is negligible reflectance from the skin at such wavelengths). This range is often referred to as far infrared energy. In one example, the wavelengths define Infrared-C (IR-C) energy.

The skin absorbs IR-C energy in the outer layers of skin (e.g., the epidermis and/or the outer 100-500 microns of skin). As a result, heating devices of the present disclosure will heat these outer layer, with deeper heating occurring via conduction through the tissue and via convection to skin blood flow. One advantage of IR-C radiation is that this type of radiation is remarkably safe, i.e., it is heat and, importantly, not a carcinogen, nor a promoter for other carcinogens, nor is it known to have any other deleterious effect on living tissue. The only known complication associated with IR-C radiation exposure is that if one allows their skin temperature to exceed 42° C. for an extended time period, then an erythema (redness of the skin due to capillary dilation) can develop. Standards have been established by the International Commission for Non-Ionizing Radiation (ICNIRP) for human exposure to IR-C radiation, and for exposures lasting 1000 seconds or longer, exposures levels are required to be kept to less than 100 W/m²—which is approximately ten times the radiation level required to remedy heat imbalances of an individual with a body surface area of about 2 m².

While 5 W-20 W of heat imbalance appears to be a small imbalance, it is not a simple task to provide radiant heating to compensate for this negative balance using traditional heat sources. To radiate effectively in the 3-10 micron range, a surface needs to be at 100°-400° C., and to ensure that much of the human body is exposed to such a heat source, the heater must have a large surface area. The size and cost of operation for such devices makes it impractical to pursue such a strategy in most commercial situations (though this is the essence of a sauna), and radiant supplemental heating is therefore not commonly employed in buildings.

However, in one example, a CO₂ laser provides a very inexpensive and very effective radiant energy source which can supply 100% of its energy in the IR-C range. Exemplary CO₂ lasers are the least expensive lasers to build, their energy output is at a wavelength of 10.4 microns, and this energy can be aimed at a target far from the source, and as importantly the beam can be easily “defocused” to provide a region of coverage which encompasses the full body of a person, yet without wasting energy output in heating the surrounding infrastructure. Recent advances in the medical and industrial use of CO₂ lasers has resulted in a dramatic decrease in size and cost for lasers delivering up to 50 W of power.

The development of a supplemental personal heating system and thermal/temperature control technology could therefore serve to keep workers in neutral heat balance when working in the typical office environment, without resorting to the use of inefficient space heaters. The technology would allow an individual to obtain the specific comfort level they desire, thereby resulting in improved productivity and decreased building operational costs.

While personal heating and thermal/temperature control technology has the potential to improve employee comfort and productivity, while lowering facility operating costs, the embodiments can be applied to cause another unexpected, very interesting, and very useful, physiologic response associated with the type of radiant heating technology that is the subject of this disclosure. Specifically, daily, transient increases in the skin surface of a person to about 39° C. can result in a rapid, and significant reduction in body weight.

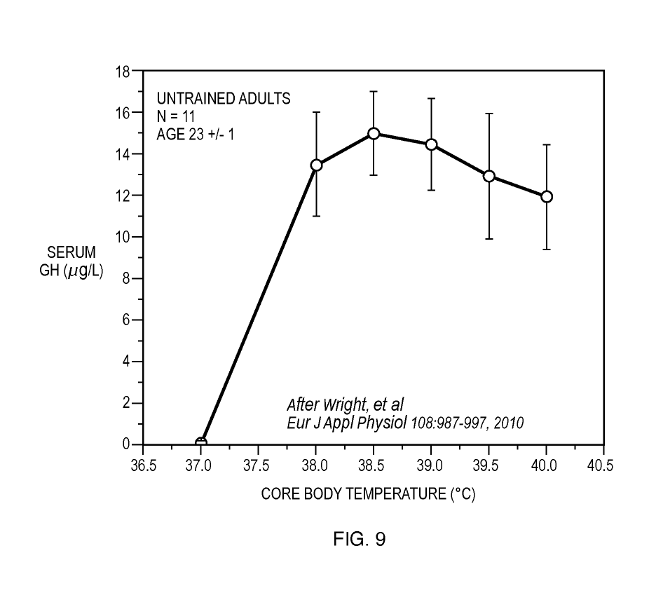

As shown in the plot of FIG. 9, one of the primary regulators of body mass is the growth hormone, and the production of growth hormone (GH) is strongly dependent on core body temperature. A transient rise in core body temperature to about 39° C. results in more than a 100× increase in GH synthesis in the body.

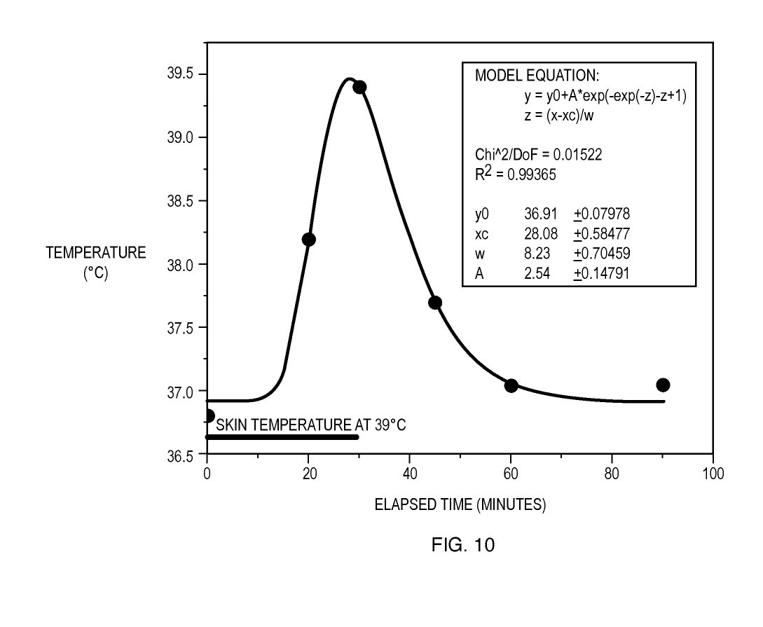

FIG. 10 shows a plot that illustrates that raising the skin temperature of the body to 39° C. for just 30 minutes results in core body temperature rise to over about 39° C. Upon termination of the exposure, core body temperature rapidly returns to “normal” levels, such that the rise is core body temperature is transient.

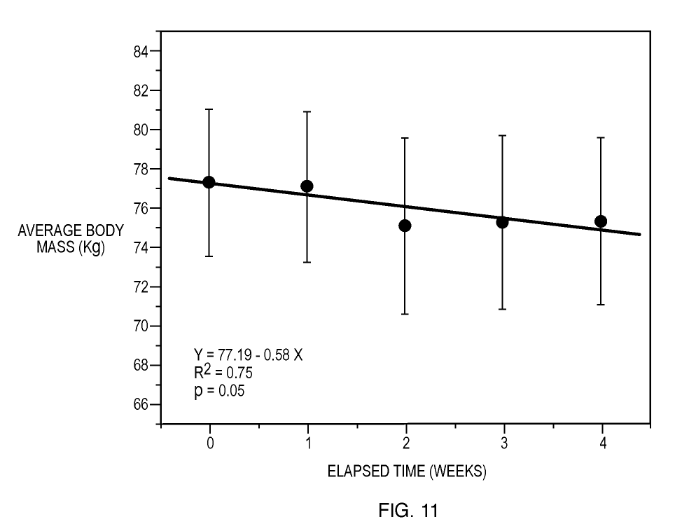

As best shown in FIG. 11, based on the ease with which short-term changes in skin temperature can influence core body temperatures, an intervention study was initiated in which a population of young adult women with a body mass index (BMI) greater than 24 Kg/m² volunteered to undergo thermal “treatment.” Three times a week, these women underwent 30 minute exposures which took their skin temperature to about 39° C. Within four weeks, 80% of these women lost body weight ranging from 1-9 pounds, with those most compliant with the “treatment” losing the greatest weight. This represented a significant loss of weight as a function of time (1.25 pounds per week, p=0.05; Figure).

Note that, even though the core body temperature rise under the exposure situation utilized was only transient, there is a clear and significant long term effect of the exposure. As the average body mass of the study population was 77 Kg, the observed average 2 Kg decrease in body mass over three weeks represents a 3% drop in body mass over just four weeks. Further, these experimental results were obtained with three times per week exposure for thirty minutes. But such a high temperature, brief exposure is not necessary to achieve similar results. As seen in FIG. 8, even a 0.5° C. rise in core body temperature can result in a 50-100 fold increase in GH production, with a correspondingly influence on body weight. Such an effect on core body temperature could be obtained by bringing skin temperature up to just above 35° C. While the rise in GH will be lower, an employee would typically be experiencing this GH stimulus each work day, for extended time periods, resulting in an accumulated effect.

### Implementation

As set forth herein, embodiments of the various control and processing devices (e.g., control device 332 of FIG. 4) can comprise processors, memory, and/or computers and computing devices with processors and memory, that can store and execute certain executable instructions, software programs, and the like. These control devices can be a separate unit, e.g., part of a control unit that operates a heating device and/or other equipment that can operate on a system level, e.g., to regulate environmental conditions in a building setting. In other examples, these control devices integrate with the heating device, e.g., as part of the hardware and/or software configured on such hardware. In still other examples, these control devices can be located remote from the heating device, e.g., in a separate location where the control device can issue commands and instructions using wireless and wired communication via a network (e.g., network 354 of FIG. 4).

The control devices may have constructive components, for example, can communicate amongst themselves and/or with other circuits (and/or devices), which execute high-level logic functions, algorithms, as well as executable instructions (e.g., firmware and software instructions and programs). Exemplary circuits of this type include, but are not limited to, discrete elements such as resistors, transistors, diodes, switches, and capacitors. Examples of processor(s) include microprocessors and other logic devices such as field programmable gate arrays (“FPGAs”) and application specific integrated circuits (“ASICs”). Although all of the discrete elements, circuits, and devices function individually in a manner that is generally understood by those artisans that have ordinary skill in the electrical arts, it is their combination and integration into functional electrical groups and circuits that generally provide for the concepts that are disclosed and described herein.

The structure of the components in the control devices can permit certain determinations, for example, as to the properties the energy beam and/or other operating parameters of the heating device 100. For example, the electrical circuits of the control device can physically manifest theoretical analysis and logical operations and/or can replicate in physical form an algorithm, a comparative analysis, and/or a decisional logic tree, each of which operates to assign the output and/or a value to the output that correctly reflects one or more of the nature, content, and origin of the changes in the beam properties that are to occur and that are reflected by the relative inputs to the control devices, e.g., as provided by temperature sensors.

In one embodiment, the processor is a central processing unit (CPU) such as an ASIC and/or an FPGA that is configured to instruct and/or control operation of the energy source. This processor can also include state machine circuitry or other suitable components capable of controlling operation of the components as described herein. The memory includes volatile and non-volatile memory and can store executable instructions including software (or firmware) instructions and configuration settings. Various other circuitry can embody stand-alone devices such as solid-state devices. Examples of these devices can mount to substrates such as printed-circuit boards and semiconductors, which can accommodate various components including the processor, the memory, and other related circuitry to facilitate operation of the control device in connection with its implementation in the heating device.

However, although the processor, the memory, the components of the control device may be configured as discrete circuitry and combinations of discrete components, this need not be the case. For example, one or more of these components can comprise a single integrated circuit (IC) or other component. As another example, the processor can include internal program memory such as RAM and/or ROM. Similarly, any one or more of functions of these components can be distributed across additional components (e.g., multiple processors or other components).

As will also be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

As used herein, an element or function recited in the singular and proceeded with the word “a” or “an” should be understood as not excluding plural said elements or functions, unless such exclusion is explicitly recited. Furthermore, references to “one embodiment” of the claimed invention should not be interpreted as excluding the existence of additional embodiments that also incorporate the recited features.

This written description uses examples to disclose embodiments of the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

In light of the forgoing discussion, this disclosure contemplates various embodiments of the heating device, as well as systems and methods consistent therewith, a sample of which includes:

A1. In one embodiment, a heating device comprising an energy source that can generate an energy beam having beam properties that, when absorbed in outer layers of skin of an end user, changes the outer layer from a first temperature to a second temperature, wherein the second temperature is higher relative to the second temperature.

A2. In one embodiment of the heating device of A1, the energy source comprises a CO₂ laser.

A3. In one embodiment of the heating device of A, further comprising a control component to operate the energy source, where in the control component comprises a processor, memory, and executable instructions stored on memory and configured to be executed by the processor.

A4. In one embodiment of the heating device of A1, further comprising a diffuser that receives energy from the energy source, wherein the diffuser has a form factor to form the energy beam.

A5. In one embodiment of the heating device of A1, wherein the beam properties include a wavelength of 3 micrometers or greater.

A6. In one embodiment of the heating device of A1, wherein the energy source generates energy with a wavelength of about 10.5 micrometers or greater.

B1. A system comprising a heating device that an energy beam having beam properties that, when absorbed in outer layers of skin of an end user, changes the outer layer from a first temperature to a second temperature and a peripheral device that couples with the heating device, wherein the peripheral device provides an input to the heating device, and wherein the heating device changes the beam properties in response to the input.

B2. In one embodiment of the system of B1, wherein the peripheral device comprises a temperature sensor disposed on the outer layers of skin of the end user.

B3. In one embodiment of the system of B1, wherein the peripheral device comprises a remote control,

B4. In one embodiment of the system of B1, wherein the input is instructive of operation of the energy source.

B5. In one embodiment of the system of B1, wherein the input is instructive of operation of the heating device.

C1. A method for changing the temperature of outer layers of skin on an end user, the method comprising receiving an input from a peripheral device, identifying the source of the input, generating an output that instructs operation of an energy source, wherein the output instructs the energy source to change one or more properties of an energy beam that impinges on the outer layers of skin of the end user.

C2. In one embodiment of the method of C1, wherein the output changes the intensity of the energy beam.

C3. In one embodiment of the method of C1, wherein the output changes the area of coverage of the energy beam.

C4. In one embodiment of the method of C1, wherein the output turns the energy source on or off.

D1. A method to promote weight loss, the method comprising irradiating an end user with an energy beam, wherein the energy beam has a wavelength in the far infrared spectrum.

D2. In one embodiment of the method of D1, wherein the wavelength defines an IR-C energy beam.

D3. In one embodiment of the method of D1, further comprising monitoring a temperature for outer layers of skin of the end user, wherein the power output of the energy beam is selected to cause the temperature to reach about 30° C. to about 38° C.

D4. In one embodiment of a method of D1, wherein the end user is irradiated for a set time period and at a periodic interval.

What is claimed is:

1. A device for heating a target, comprising:
an energy source component adapted to generate an energy beam having a wavelength in an infrared spectrum of electromagnetic energy; and
an optics component in position relative to the energy source to receive the energy beam, the optics component comprising a lens element with a first side and a second side that is closer to the target than the first side,
wherein the energy beam has a first beam configuration on the first side of the lens element and a second beam configuration on the second side of the lens element that define, respectively, a first coverage region and a second coverage region that is larger than the first coverage region.

2. The device of claim 1, wherein the energy beam has a wavelength of 3 micrometers or larger.

3. The device of claim 1, wherein the energy source comprises a CO₂ laser.

4. The device of claim 1, wherein the energy source has a power rating in a range from about 10 W to about 50 W.

5. The device of claim 1, wherein the wavelength defines a far infrared spectrum.

6. The device of claim 1, wherein the second beam configuration defines a curvilinear shape.

7. The device of claim 6, wherein the curvilinear shape forms an ellipse.

8. A system for heating a target, said system comprising:
a heating device for generating an energy beam with a wavelength in an infrared spectrum of electromagnetic energy, the energy beam having a beam configuration that defines a coverage region at the target to modify temperature of the target from a first temperature to a second temperature that is higher than the first temperature; and
a control device that is configured to generate an output to instruct operation of the heating device,
wherein the coverage region has a shape with an area of 500 cm² or greater at the target.

9. The system of claim 8, further comprising a sensor element coupled with the control device, wherein the sensor element is configured to generate a signal that defines a value for a target response parameter of the target at the second temperature.

10. The system of claim 9, wherein the control device comprises a processor, memory, and executable instructions stored on the memory and configured to be executed by the processor, and wherein the executable instructions comprise instructions for:
receiving the signal from the sensor device; and
generating the output to instruct the heating device to modulate the energy beam in response to the value.

11. The system of claim 9, wherein the target response parameter identifies a temperature for a layer of skin on the target.

12. The system of claim 9, wherein the target response parameter identifies a response of the target at the second temperature.

13. The system of claim 12, wherein the response measures a physiological response of the target.

14. The system of claim 13, wherein the physiological response identifies a change in weight of a human from a first weight to a second weight that is different from the first weight.

15. The system of claim 12, wherein the response measures a clinical response of a human during a surgical procedure.

16. The system of claim 15, wherein the clinical response comprises neurological activity of the human.

17. The system of claim 16, further comprising a device that couples with the human to record electrical activity of the human that indicates the neurological activity.

18. The system of claim 8, wherein the control device comprises a remote control that communicates wirelessly with the heating device.

19. A method of heating a human, comprising:
irradiating the human with an energy beam having a wavelength in an infrared spectrum to modify temperature of the human from a first temperature to a second temperature that is higher than the first temperature;
monitoring a value for a target response parameter at the second temperature;
comparing the value to a threshold criteria for the target response parameter; and
modulating the energy beam when the value satisfies the threshold criteria to change the temperature of the target from the second temperature.

20. The method of claim 19, wherein the target response parameter identifies a temperature for a layer of skin on the human.

21. The method of claim 19, wherein the target response parameter identifies a physiological response of the human to the second temperature.

22. The method of claim 21, wherein the physiological response comprises a biochemical response of the human.

23. The method of claim 22, wherein the biochemical response measures a level of human growth hormone of the human.

24. The method of claim 19, wherein the target response parameter identifies a clinical response of the human at the second temperature

25. The method of claim 24, wherein the clinical response measures neurological activity of the human.

26. The method of claim 19, wherein the target response parameter and the threshold criteria define an exposure parameter for irradiation of the human.

27. The method of claim 26, further comprising:
collecting a first body indicator of the human; and
comparing the first body indicator to a baseline value for the human,
wherein the exposure parameter defines a period of time that the human is subject to irradiation by the energy beam.

28. The method of claim 27, wherein the first body indicator and the baseline value define a weight for the human.

29. The method of claim 27, wherein the first body indicator and the baseline value define a percent body fat for the human.
